(19) United States
(12) Patent Application Publication
(10) Pub. No.: US 2026/0038693 A1
(43) Pub. Date: Feb. 5, 2026

(54) INFORMATION PROCESSING DEVICE, INFORMATION PROCESSING METHOD, AND PROGRAM

(71) Applicant: NEC Corporation, Tokyo (JP)

(72) Inventor: Terumi UMEMATSU, Tokyo (JP)

(73) Assignee: NEC Corporation, Tokyo (JP)

(21) Appl. No.: 19/279,018

(22) Filed: Jul. 24, 2025

(30) Foreign Application Priority Data
Jul. 30, 2024 (JP) 2024-123351

(51) Int. Cl. G16H 50/30; A61B 5/00; A61B 5/16; G06Q 40/08; G06T 7/00

(52) U.S. Cl. CPC G16H 50/30; A61B 5/165; A61B 5/4064; A61B 5/7275; G06Q 40/086; G06T 7/0016; G06T 2207/10016; G06T 2207/30004; G06T 2207/30201

(57) ABSTRACT

In an information processing device, a face video data acquisition means acquires a face video of the subject. A disease risk estimation means estimates a disease risk score including a mental disease risk score, a brain disease risk score, and a physical disease risk score based on the face video of the subject. A proposal means decides a proposal content for the subject based on a combination of the mental disease risk score, the brain disease risk score, and the physical disease risk score. The information processing device can support optimal decision-making regarding the provision of services and products.

### INCORPORATION BY REFERENCE

This application is based upon and claims the benefit of priority from Japanese patent application No. 2024-123351, filed on Jul. 30, 2024, the disclosure of which is incorporated herein in its entirety by reference.

### TECHNICAL FIELD

The present disclosure relates to health condition estimation.

### BACKGROUND ART

Life insurance companies often set uniform life insurance premiums for insurance contractors. However, since the health condition varies depending on the person, it is not necessarily appropriate to uniformly set the life insurance premium for all the insurance contractors. Patent Document 1 discloses an insurance premium setting system that changes an insurance premium according to a smile in consideration of contribution to health by the smile.
Patent Document 1: WO 2016/178327 A1

### SUMMARY

However, it is not always possible to set an optimized fee for each person also by the method for Patent Document 1.

One object of the present disclosure is to provide an information processing device capable of making an optimal proposal according to the health condition of each person.

### Means for Solving the Problem

According to an example aspect of the present disclosure, there is provided an information processing device, comprising:
at least one memory configured to store instructions; and
at least one processor configured to execute the instructions to:
acquire a face video of a subject;
estimate a disease risk score including a mental disease risk score, a brain disease risk score, and a physical disease risk score based on a face video of the subject; and
decide a proposal content for the subject based on a combination of the mental disease risk score, the brain disease risk score, and the physical disease risk score.

According to a further example aspect of the present disclosure, there is provided an information processing method executed by a computer, the method comprising: acquiring a face video of a subject;
estimating a disease risk score including a mental disease risk score, a brain disease risk score, and a physical disease risk score based on a face video of the subject; and
deciding a proposal content for the subject based on a combination of the mental disease risk score, the brain disease risk score, and the physical disease risk score.

According to a further example aspect of the present disclosure, there is provided a recording medium recording a program for causing a computer to execute processing comprising:
acquiring a face video of a subject;
estimating a disease risk score including a mental disease risk score, a brain disease risk score, and a physical disease risk score based on a face video of the subject; and
deciding a proposal content for the subject based on a combination of the mental disease risk score, the brain disease risk score, and the physical disease risk score.

### Effect

According to the present disclosure, it is possible to make an optimal proposal according to the health condition of each person.

### BRIEF DESCRIPTION OF THE DRAWINGS

FIG. 1 is a diagram illustrating an overall configuration of a health management system according to the present disclosure;

FIG. 2 is a block diagram illustrating a hardware configuration of an information processing device according to the present disclosure;

FIG. 3 is a block diagram illustrating a functional configuration of an information processing device according to the present disclosure;

FIG. 4 is an example of a selection table;

FIG. 5 is a flowchart of disease risk estimation processing;

FIG. 6 is an example of health items of an insured person;

FIG. 7 is a block diagram illustrating a functional configuration of another information processing device according to the present disclosure;

FIG. 8 is a diagram conceptually illustrating another health management system according to the present disclosure;

FIG. 9 is a block diagram illustrating a functional configuration of another information processing device according to the present disclosure; and

FIG. 10 is a flowchart of processing by another information processing device according to the present disclosure.

### EXAMPLE EMBODIMENT

Hereinafter, example embodiments of the present disclosure will be described with reference to the drawings.

### First Example Embodiment

### [Overall Configuration]

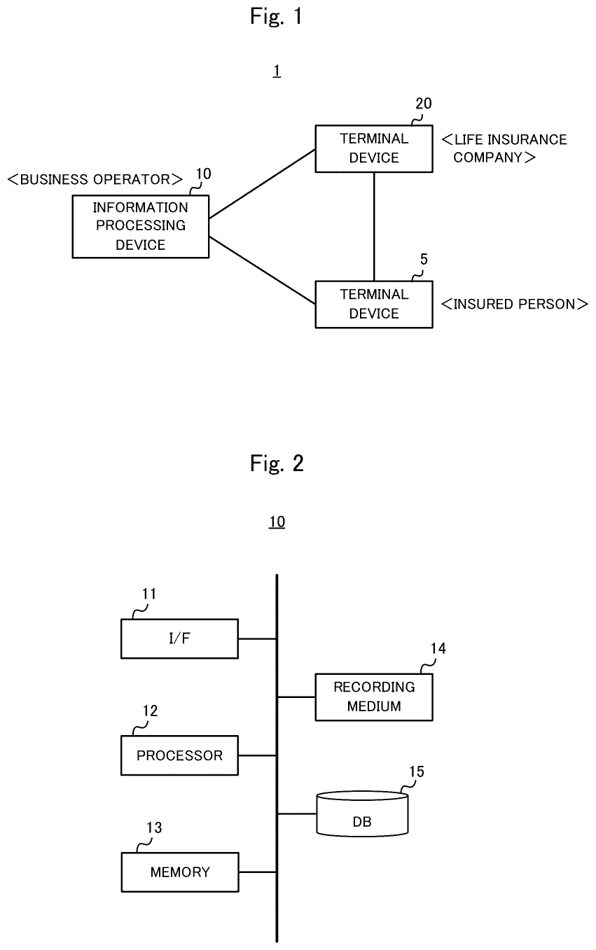

FIG. 1 illustrates an overall configuration of a health management system to which an information processing device according to the present disclosure is applied. A health management system 1 includes a terminal device 5 of an insured person, an information processing device 10, and a terminal device 20 of a life insurance company.

The terminal device 5 of the insured person is operated by the insured person or the like, and is used to photograph the face of the insured person. The terminal device 5 may be constituted by, for example, a smartphone or a tablet terminal owned by an insured person, or may be constituted by a camera device or the like installed in a facility of a life insurance company. The terminal device 5 communicates with the information processing device 10 and a terminal device 20 of a life insurance company through a network such as the Internet.

The information processing device 10 estimates the health condition of the insured person from the face video of the insured person. The information processing device 10 proposes services and products related to health care such as health maintenance and health promotion based on the health condition of the insured person. The information processing device 10 reviews the insurance premium based on the health condition of the insured person. The information processing device 10 includes, for example, a server device or the like, and communicates with the terminal device 5 of the insured person or the terminal device 20 of a life insurance company through a network such as the Internet.

The terminal device 20 of the life insurance company is operated by a person in charge of the life insurance company or the like. The terminal device 20 may execute some processes executed by the information processing device 10. Specifically, the terminal device 20 can receive the health condition of the insured person from the information processing device 10, and review the service proposal, the product proposal, and the insurance premium. The terminal device 20 includes, for example, a personal computer, a server device, or the like, and communicates with the terminal device 5 of the insured person or the information processing device 10 through a network such as the Internet.

### [Hardware Configuration]

FIG. 2 is a block diagram illustrating a hardware configuration of the information processing device 10 according to the first example embodiment. As illustrated, the information processing device 10 includes an interface (I/F) 11, a processor 12, a memory 13, a recording medium 14, and a database (DB) 15.

The I/F 11 communicates with the terminal device 5 of the insured person and the terminal device 20 of the life insurance company through a network such as the Internet.

The processor 12 is a computer such as a central processing unit (CPU), and controls the entire information processing device 10 by executing a program prepared in advance. As the processor 12, for example, a graphics processing unit (GPU), a digital signal processor (DSP), a micro processing unit (MPU), a floating point number processing unit (FPU), a physics processing unit (PPU), a tensor processing unit (TPU), a quantum processor, a combination thereof, or the like can be used. The processor 12 executes disease risk estimation processing to be described later.

The memory 13 includes a read only memory (ROM), a random access memory (RAM), and the like. The memory 13 is also used as a working memory during execution of various processes by the processor 12.

The recording medium 14 is a non-volatile non-transitory recording medium such as a disk-shaped recording medium or a semiconductor memory, and is configured to be attachable to and detachable from the information processing device 10. The recording medium 14 records various programs executed by the processor 12. If the information processing device 10 executes various types of processing, a program recorded in the recording medium 14 is loaded into the memory 13 and executed by the processor 12.

For example, the DB 15 may store information regarding the insured person in association with the insured person ID. The DB 15 may store the past face video and the past health condition of the insured person in association with the insured person ID. The insured person ID is an ID for uniquely identifying the insured person.

In addition to the above, the information processing device 10 may include a display device such as a liquid crystal display and an input device such as a keyboard and a mouse. These display devices and input devices are used, for example, by an administrator of the information processing device 10 to perform necessary management.

### [Functional Configuration]

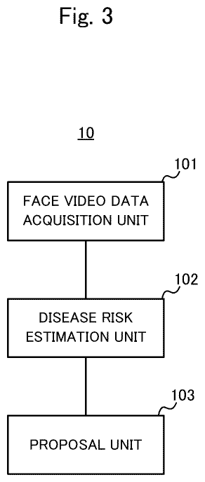

FIG. 3 is a block diagram illustrating a functional configuration of the information processing device 10 according to the first example embodiment. The information processing device 10 functionally includes a face video data acquisition unit 101, a disease risk estimation unit 102, and a proposal unit 103.

The face video data acquisition unit 101 acquires the face video of the insured person photographed by the terminal device 5. The face video data acquisition unit 101 outputs the face video to the disease risk estimation unit 102.

The disease risk estimation unit 102 estimates the health condition of the insured person. In the present example embodiment, a disease risk score obtained by scoring a disease risk is used as an index indicating the health condition of the insured person. In the present example embodiment, the disease risk score is represented by binary values of 0 (low risk) and 1 (high risk). Hereinafter, the fact that the disease risk score is 0 is also simply referred to as “low disease risk”, and the fact that the disease risk score is 1 is also simply referred to as “high disease risk”.

The disease risk estimation unit 102 determines a disease risk score based on the face video of the insured person. In the present example embodiment, the disease risk includes a mental disease risk, a brain disease risk, and a physical disease risk. The disease risk estimation unit 102 determines the score of each disease risk and outputs the determination result to the proposal unit 103.

Specifically, the disease risk estimation unit 102 estimates a value of an item related to health (hereinafter, also referred to as a “health item”) based on the face video of the insured person. Then, the disease risk estimation unit 102 determines whether each disease risk is high based on the estimation result of each item. The health item includes, for example, drowsiness and a concentration level of the insured person, stress, cognitive function, vital signs (heart rate, respiratory rate, SpO2, blood pressure, blood glucose level, cholesterol level, etc.), and the like.

### (1) Mental Disease Risk

The disease risk estimation unit 102 estimates the drowsiness, concentration level, stress, and the like of the insured person based on the face video of the insured person, and determines whether the mental disease risk is high. For example, the disease risk estimation unit 102 determines that the mental disease risk is high in a case where the drowsiness of the insured person is strong, in a case where the insured person is not concentrating, or in a case where the stress of the insured person is high. The disease risk estimation unit 102 may determine the mental disease risk based on one of the following items: drowsiness, concentration level, and stress. Alternatively, it may determine the mental disease risk by combining a plurality of items.

### (2) Brain Disease Risk

The disease risk estimation unit 102 estimates the drowsiness, concentration level, cognitive function, and the like of the insured person based on the face video of the insured person, and determines whether the brain disease risk is high. For example, the disease risk estimation unit 102 determines that the brain disease risk is high in a case where the drowsiness of the insured person is strong, in a case where the insured person is not concentrating, or in a case where the cognitive function of the insured person is low. The disease risk estimation unit 102 may determine the brain disease risk based on one of the following items: drowsiness, concentration level, and cognitive function. Alternatively, it may determine the brain disease risk by combining a plurality of items.

### (3) Physical Disease Risk

The disease risk estimation unit 102 estimates drowsiness, vital signs (heart rate, respiratory rate, SpO2, blood pressure, blood glucose level, cholesterol level, etc.), and the like of the insured person based on the face video of the insured person, and determines whether the physical disease risk is high. For example, the disease risk estimation unit 102 determines that the physical disease risk is high in a case where the drowsiness of the insured person is strong or in a case where the vital sign is an abnormal value. The disease risk estimation unit 102 may determine the physical disease risk based on one of the following items: drowsiness or an individual vital sign. Alternatively, it may determine the physical disease risk by combining a plurality of items.

In the above (1) to (3), in a case where the disease risk is determined by combining a plurality of items, the disease risk estimation unit 102 scores the estimation results for the plurality of items based on a predetermined criterion. Then, the disease risk estimation unit 102 determines whether the disease risk is high according to the total value, the average value, or the like of the scores. The disease risk estimation unit 102 may determine whether the disease risk is high using a machine learning model. This machine learning model is, for example, a machine learning model learned in advance such that estimation results for a plurality of items are input and a disease risk score is output.

The disease risk estimation unit 102 outputs the determination results of disease risks obtained by (1) to (3) described above to the proposal unit 103.

The disease risk estimation unit 102 may represent the disease risk score not as a binary of 0 and 1 but as a continuous value indicating the degree of disease risk. For example, in a case where the range of the continuous value is 0 to 100, it is assumed that the closer to 100, the higher the disease risk.

The proposal unit 103 makes a proposal suitable for the insured person based on the determination result of each disease risk. For example, the proposal unit 103 selects a service suitable for the insured person from among a plurality of services prepared in advance. The proposal unit 103 selects an insurance product suitable for the insured person from among a plurality of insurance products prepared in advance.

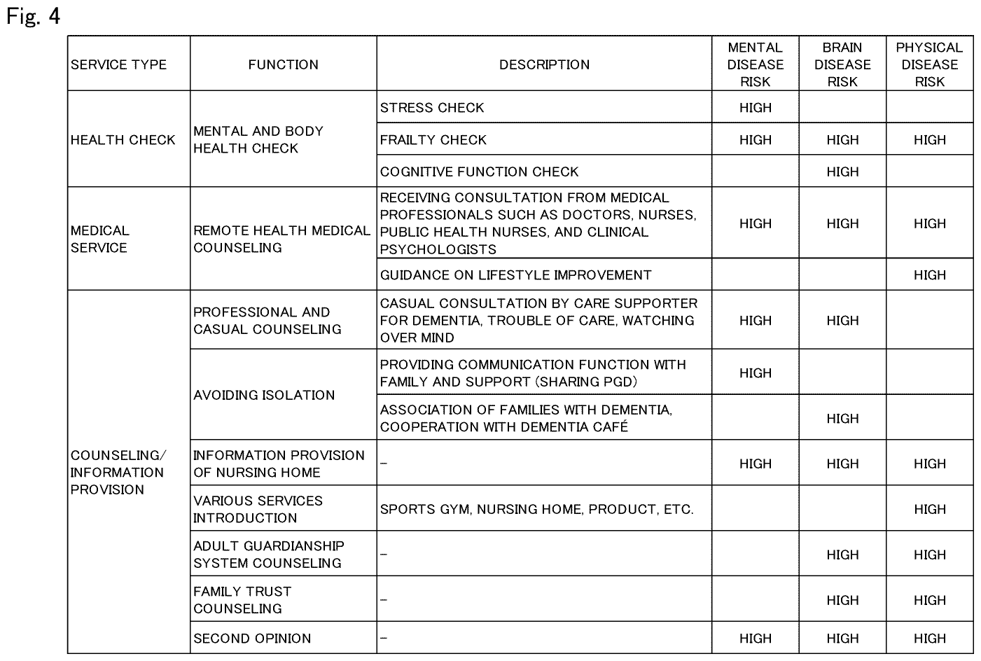

Specifically, the proposal unit 103 selects a service based on the determination result of each disease risk. FIG. 4 illustrates a service selection table. In the selection table of FIG. 4, a relationship between a service and a combination of disease risks is determined. Regarding the disease risk, “high” indicates that the disease risk is high.

The proposal unit 103 selects a service based on a combination of the disease risk determination results with reference to the table illustrated in FIG. 4. For example, in a case where all of the three disease risks are high, the proposal unit 103 selects “frailty check”, “acceptance of consultation by medical professional”, “information provision of nursing home”, and “second opinion”. In a case where there are a plurality of services as described above, the proposal unit 103 may select at least one or more services.

Regarding the disease risk in FIG. 4, a blank indicates that the disease risk is low. For example, in a case where the mental disease risk is high and the brain disease risk and the physical disease risk are low, the proposal unit 103 selects “stress check” and “communication function provision”.

Regarding the disease risk in FIG. 4, blanks may indicate that the disease risk may be high or low. For example, if the mental disease risk is high, the proposal unit 103 may select “stress check” as the proposed service regardless of the levels of the brain and physical disease risks.

The proposal unit 103 selects an insurance product based on a combination of the disease risk determination results. For example, the proposal unit 103 selects a health promotion type insurance in a case where all the mental, brain, and physical disease risks are low, selects a dementia insurance in a case where the brain disease risk is high, and selects a care insurance in a case where any of the mental, brain, and physical disease risks is high.

The proposal unit 103 outputs the proposal content to the terminal device 20 of the life insurance company. A person in charge of a life insurance company can consider appropriate health support and counseling for an insured person by referring to the proposal content.

For example, the proposal unit 103 controls display to show the proposal content on the display of the terminal device 20. The proposal unit 103 select a terminal device for display control based on the proposal content among a plurality of terminal devices, and then perform display control of the proposal content. Specifically, the proposal unit 103 refers to the IDs of terminal devices permitted to display for each proposal content in the DB, refers to the IDs of multiple communicable terminal devices 20, and automatically selects a terminal device for display control from among them. Even when the number of terminal devices to be controlled is enormous, real-time display control to only appropriate terminal devices becomes possible.

In the above configuration, the face video data acquisition unit 101 is an example of a face video data acquisition means, the disease risk estimation unit 102 is an example of a disease risk estimation means, and the proposal unit 103 is an example of a proposal means.

### [Processing Flow]

Next, the disease risk estimation processing by the information processing device 10 will be described.

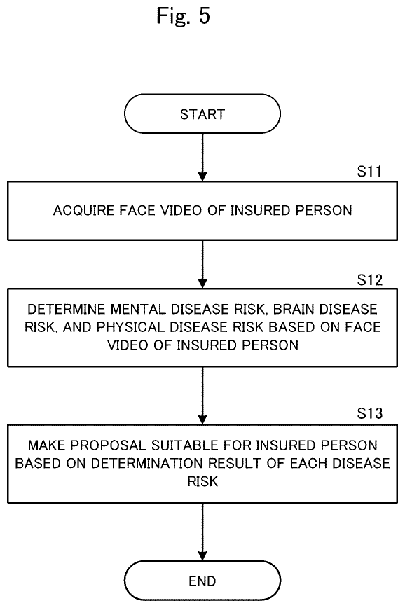

FIG. 5 is a flowchart of disease risk estimation processing by the information processing device 10. This processing is achieved by the processor 12 illustrated in FIG. 2 executing a program prepared in advance and operating as each element illustrated in FIG. 3.

First, the face video data acquisition unit 101 acquires the face video of the insured person photographed by the terminal device 5 (step S11). The face video data acquisition unit 101 outputs the face video to the disease risk estimation unit 102.

Next, the disease risk estimation unit 102 determines a mental disease risk, a brain disease risk, and a physical disease risk based on the face video of the insured person (step S12). Specifically, the disease risk estimation unit 102 estimates drowsiness, a concentration level, stress, cognitive function, vital signs, and the like of the insured person. Then, the disease risk estimation unit 102 determines whether each disease risk is high based on the estimation result. The disease risk estimation unit 102 outputs a determination result of each disease risk to the proposal unit 103.

Next, the proposal unit 103 makes a proposal suitable for the insured person based on the determination result of each disease risk (step S13). The proposal unit 103 outputs the proposal content to the terminal device 20 of the life insurance company. Then, the process ends.

### [Method for Estimating Health Item]

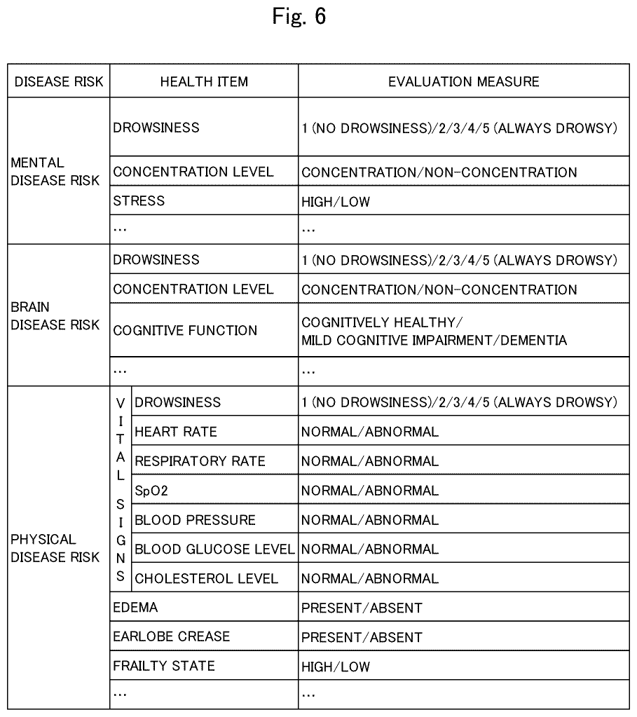

Next, a method for estimating the value of the health item of the insured person will be described. FIG. 6 is an example of health items of the insured person, and includes the risk of edema, wrinkles, and frailty in addition to the drowsiness and concentration level of the insured person, stress, cognitive function, and vital signs.

### (1) Estimation of Drowsiness

The disease risk estimation unit 102 detects movement of the eyelid of the insured person from the face video of the insured person, and estimates drowsiness based on the detected movement. The drowsiness is expressed in five levels, and the higher the numerical value, the stronger the drowsiness. A method for estimating the drowsiness from the face video is described in the following document, for example. The following documents are incorporated herein as references.

M. Tsujikawal, Y. Onishil, Y. Kiuchil, T. Ogatsul, A. Nishino and S. Hashimoto, “Drowsiness Estimation from Low-Frame-Rate Facial Videos using Eyelid Variability Features,” 2018 40th Annual International Conference of the IEEE Engineering in Medicine and Biology Society (EMBC), Honolulu, HI, USA, 2018, pp. 5203-5206.

In a case where the drowsiness of the insured person is strong, the disease risk estimation unit 102 determines that the mental disease risk, the brain disease risk, and the physical disease risk are high. The disease risk estimation unit 102 may determine the disease risk in consideration of the time when the face video is captured. For example, in a case where the drowsiness in the daytime (13:00 to 15:00) is strong, the disease risk estimation unit 102 determines that the mental disease risk and the physical disease risk are high.

### (2) Estimation of Concentration Level

The disease risk estimation unit 102 detects the movement of the eyelid, the line of sight, the expression, and the like of the insured person from the face video of the insured person, and estimates the concentration level based on the detected movement. The concentration level is expressed in two stages of concentration and non-concentration. A method for estimating the concentration level from the face video is described in, for example, the following document. The following documents are incorporated herein as references.

Terumi Umematsu, Masanori Tsujikawa, Hideyuki Sawada, “Evaluation of Cognitive Test Results Using Concentration Estimation from Facial Videos,” Asia-Pacific Signal and Information Processing Association Annual Summit and Conference (APSIPA), pp. 261-266, November 2022.

In a case where the insured person is not concentrating (non-concentration), the disease risk estimation unit 102 determines that the mental disease risk and the brain disease risk are high. The disease risk estimation unit 102 may determine the disease risk in consideration of the time when the face video is captured or the report of the insured person. For example, in a case where the insured person is not concentrating during operating or working, the disease risk estimation unit 102 determines that the mental disease risk and the brain disease risk are high.

### (3) Estimation of Stress

The disease risk estimation unit 102 measures heart rate variability from the face video of the insured person by a remote photoplethysmography (rPPG) technology and calculates a stress index (LF/HF). The method for measuring the heart rate variability from the face video is described in the following document, for example. The following documents are incorporated herein as references.

Terumi Umematsu and Masanori Tsujikawa, “Heart rate estimation from facial videos based on ICA with reference”, The 39th International Conference of the IEEE Engineering in Medicine and Biology Society (EMBC), July 2017.

The following document proposes a stress estimation method using a contactless heart rate measurement method from a face video. The following documents are incorporated herein as references.

Asami Umematsu, Takanori Tsujigawa, and Hideyuki Sawada, “Real-time stress estimation using contactless heart rate measurement robust to facial movements,” Journal of Information Processing Society of Japan, Vol. 65, No. 7, pp. 1150-1161, 2024.

In a case where the stress is continuously high, the disease risk estimation unit 102 determines that the mental disease risk is high. Specifically, in a case where LF/HF is always higher than the reference value (sympathetic nerve is always dominant) or in a case where LF/HF is not lower than the reference value (parasympathetic nerve is not dominant), the disease risk estimation unit 102 determines that the mental disease risk is high.

### (4) Estimation of Cognitive Function

The disease risk estimation unit 102 calculates the eye closing ratio and the eyelid movement speed of the subject from the face video of the insured person, and estimates the cognitive function based on the calculated values. The disease risk estimation unit 102 may express the cognitive function in three stages of “cognitive health”, “mild cognitive impairment”, and “dementia”, or may express the cognitive function in a score equivalent to the mini mental state examination (MMSE) which is one of the evaluations of the cognitive function. The method for estimating the cognitive function from the face video is described in, for example, International Application No. PCT/JP2023/041209 filed by the present inventor earlier. The description of the specification of this application is incorporated herein.

In a case where the cognitive function is low, that is, in a case of “mild cognitive impairment” or “dementia”, the disease risk estimation unit 102 determines that the brain disease risk is high.

### (5) Vital Sign Estimation

In FIG. 6, vital signs include heart rate, respiratory rate, SpO2, blood pressure, blood glucose level, and cholesterol.

### (5)-1. Heart Rate

The disease risk estimation unit 102 estimates the heart rate by calculating a brightness change of the complexion from the face video of the insured person. A method for estimating the heart rate from the face video is described in, for example, the following document. The following documents are incorporated herein as references.

Terumi Umematsu and Masanori Tsujikawa, “Heart rate estimation from facial videos based on ICA with reference,” The 39th International Conference of the IEEE Engineering in Medicine and Biology Society (EMBC), July 2017.

The disease risk estimation unit 102 compares the estimated heart rate with a preset reference value to determine whether the heart rate is normal or abnormal. In a case where the determination result is abnormal, the disease risk estimation unit 102 determines that the physical disease risk is high. The disease risk estimation unit 102 may determine the disease risk based on the resting heart rate of the insured person. In this case, if the resting heart rate of the insured person is higher than the reference value, the disease risk estimation unit 102 determines that the physical disease risk is high.

The disease risk estimation unit 102 can estimate SpO2, blood pressure, blood glucose level, and the like from the brightness change of the complexion.

### (5)-2. Respiratory Rate

The disease risk estimation unit 102 estimates respiratory rate from the face video of the insured person using a respiratory rate (RR) estimation model prepared in advance. A method for estimating respiratory rate from a face video is described in, for example, the following document. The following documents are incorporated herein as references.

Akamatsu, Yusuke, Terumi Umematsu and Hitoshi Imaoka. “CalibrationPhys: Self-Supervised Video-Based Heart and Respiratory Rate Measurements by Calibrating Between Multiple Cameras,” IEEE Journal of Biomedical and Health Informatics 28 (2023): 1460-1471.

The disease risk estimation unit 102 compares the estimated respiratory rate with a preset reference value to determine whether the respiratory rate is normal or abnormal.

In a case where the determination result is abnormal, the disease risk estimation unit 102 determines that the physical disease risk is high.

### (5)-3. Cholesterol

The disease risk estimation unit 102 estimates BMI from the face video of the insured person using a BMI prediction model prepared in advance or the like. Then, the disease risk estimation unit 102 determines whether cholesterol is high based on BMI. In a case where the cholesterol is high, the disease risk estimation unit 102 determines that the physical disease risk (the risk of obesity) is high.

### (6) Estimation of Edema

The disease risk estimation unit 102 estimates the presence or absence of edema from the face video of the insured person using an estimation model of edema prepared in advance or the like. A method for estimating edema from a face video is described in, for example, the following document. The following documents are incorporated herein as references.

Y. Akamatsu, Y. Onishi, H. Imaoka, J. Kameyama and H. Tsurushima, “Edema Estimation From Facial Images Taken Before and After Dialysis via Contrastive Multi-Patient Pre-Training,” in IEEE Journal of Biomedical and Health Informatics, vol. 27, no. 3, pp. 1419-1430 March 2023

In a case where the insured person has edema, the disease risk estimation unit 102 determines that the physical disease risk (risk of kidney disease, heart disease, or the like) is high.

### (7) Estimation of Earlobe Crease

The disease risk estimation unit 102 estimates whether there is a crease in the earlobe of the subject (whether there is a frank's sign) by image analysis of the face image of the insured person. In a case where there is a crease on the earlobe, the disease risk estimation unit 102 determines that the physical disease risk (risk of heart disease or the like) is high.

### (8) Estimate Frailty Risk

The disease risk estimation unit 102 estimates whether there is a risk of frailty by using walking data of the insured person and the like in addition to the face video of the insured person. The method for estimating the risk of frailty is described in, for example, Japanese Patent Application No. 2024 078683 filed by the present inventor. The description of the specification of this application is incorporated herein. In a case where there is a risk of frailty, the disease risk estimation unit 102 determines that the brain and physical disease risks are high.

The method for estimating the value of the health item described above is an example, and the method is not limited thereto. The disease risk estimation unit 102 represents the value of each health item as a binary value or a stepwise value such as 3 steps or 5 steps. However, for example, the degree may be indicated by a continuous numerical value from 0 to 100.

### MODIFICATIONS

Next, modifications of the first example embodiment will be described.

### Modification 1

The information processing device 10 according to the first example embodiment selects a service or an insurance product suitable for the insured person based on the current disease risk of the insured person. In addition to the above, the information processing device 10 may select a service or an insurance product in consideration of the future disease risk of the insured person.

The face video data acquisition unit 101 periodically acquires a face video of the insured person and accumulates the acquired video. The disease risk estimation unit 102 generates a prediction model by machine learning using linear regression based on past data. Then, the disease risk estimation unit 102 predicts a future disease risk based on the prediction model. The past data includes the acquisition date and time of the face video of the insured person, the value of the health item at that time, the disease risk, and the like. The disease risk estimation unit 102 may learn a prediction model using a long short term memory (LSTM). In this case, the disease risk estimation unit 102 uses, as the past data, time-series data including the face video of the insured person and the acquisition date and time of the face video. The future prediction using the LSTM is described, for example, in the following document. The following documents are incorporated herein as references.

Terumi Umematsu, Akane Sano., and Rosalind Picard, “Daytime Data and LSTM can Forecast Tomorrow's Stress, Health, and Happiness,” Proceedings of the 41st International Conference of the IEEE Engineering in Medicine and Biology Society (EMBC), p. 2186-2190 July 2019.

The proposal unit 103 selects a service or an insurance product by using one or both of the current disease risk and the future disease risk.

### Modification 2

The information processing device 10 can determine the disease risk in more detail by combining the estimation results for a plurality of health items.

Specifically, the disease risk estimation unit 102 may evaluate each disease risk score in four stages of “lower risk”, “low risk”, “high risk”, and “higher risk” by combining estimation results for a plurality of health items. For example, regarding the combination of the drowsiness and the concentration level, the disease risk estimation unit 102 can determine that the brain and mental disease risks are higher in a case where there is no drowsiness of the insured person and the insured person is not concentrating. With respect to the combination of the drowsiness and the cognitive function, the disease risk estimation unit 102 can determine that the mental and physical disease risks are higher in a case where the drowsiness of the insured person is strong and the cognitive function of the insured person is high. Regarding the combination of the concentration level and the cognitive function, the disease risk estimation unit 102 can determine that the mental disease risk is higher in a case where the insured person is not concentrating and the cognitive function of the insured person is high.

The disease risk estimation unit 102 may narrow down the disease risk by combining estimation results of a plurality of health items. For example, in a case where the drowsiness of the insured person is strong, it can be determined that all the mental, brain, and physical disease risks are high, but in a case where the cognitive function is high even if the drowsiness is strong, it may be determined that the mental and physical disease risks are high among the three disease risks.

### Modification 3

In the first example embodiment, the life insurance company and the insured person have been described as examples of the user of the health management system, but the user of the health management system is not limited to the above. For example, instead of a life insurance company, a health insurance union, a shared use facility such as an apartment or a nursing care facility, or a commercial facility such as a convenience store may use the health management system.

In the case of the health insurance union, the insured person photographs a face video using a terminal device owned by the insured person or a camera device installed in a workplace. The information processing device 10 estimates the disease risk score based on the face video of the insured person. Then, the information processing device 10 determines whether specific health guidance is necessary based on the disease risk score. The information processing device 10 refers to the current disease risk score and the past disease risk score, and proposes health support or counseling by an expert according to a change in the score. By referring to the disease risk score and the proposal content, the person in charge of the health insurance union can provide specific health guidance, health support, and counseling to the insured person as necessary.

In the case of the shared use facility, the facility user captures a face video using a camera device installed in the facility. The information processing device 10 estimates the disease risk score based on the user's face video. Then, the information processing device 10 provides product proposals, lifestyle proposals, and guidance on events and consultation meetings based on the disease risk score. The person in charge of the shared use facility or the facility user can grasp a service or a product suitable for the facility user by referring to the disease risk score or the proposal content.

In the case of a commercial facility, a customer photographs a face video using a camera device installed in the facility. The information processing device 10 estimates the disease risk score based on the face video of the customer. Then, the information processing device 10 proposes a product, a service, or the like provided by the commercial facility based on the disease risk score. For example, in a case where the blood pressure of the customer is high, the information processing device 10 can propose a food for specified health care having an effect of lowering the blood pressure. A person in charge of a commercial facility or a customer can grasp a service or a product suitable for the customer by referring to the disease risk score or the proposal content.

The above proposal content is an example, and is not limited thereto.

### Modification 4

The information processing device 10 may include an insurance premium calculation unit 104 in addition to the face video data acquisition unit 101, the disease risk estimation unit 102, and the proposal unit 103 in the functional configuration. The insurance premium calculation unit 104 is an example of an insurance premium calculation means.

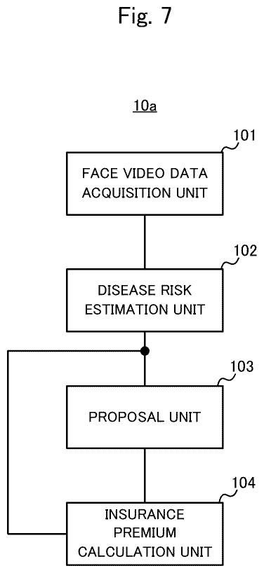

FIG. 7 is a block diagram illustrating a functional configuration of an information processing device 10a according to a second modification. The determination result of each disease risk is input from the disease risk estimation unit 102 to the insurance premium calculation unit 104, and the proposal content is input from the proposal unit 103.

The insurance premium calculation unit 104 can review the insurance premium for the insurance that the insured person has subscribed. For example, the insurance premium calculation unit 104 may decide a coefficient according to each disease risk score, and calculate the insurance premium by multiplying the current insurance premium by the coefficient. The coefficients are preset by the life insurance company. It is assumed that the life insurance company sets a coefficient relevant to a combination of each disease risk score for each insurance product. The method for calculating the insurance premium described above is an example, and the method is not limited thereto.

The insurance premium calculation unit 104 can calculate the insurance premium of the insurance product input from the proposal unit 103. For example, the insurance premium calculation unit 104 can calculate the insurance premium so that the lower the disease risk score, the lower the insurance premium, and the higher the disease risk score, the higher the insurance premium.

The insurance premium calculation unit 104 refers to the disease risk score of the insured person from the past to the present, and may make a proposal to raise the insurance premium to the life insurance company in a case where the disease risk score remains high, and may make a proposal to discount the insurance premium or give a benefit to the life insurance company in a case where the disease risk score decreases.

### Modification 5

The information processing device 10 according to the first example embodiment transmits a proposal suitable for an insured person to a life insurance company. Instead, the information processing device 10 may provide the disease risk score of the insured person to the life insurance company.

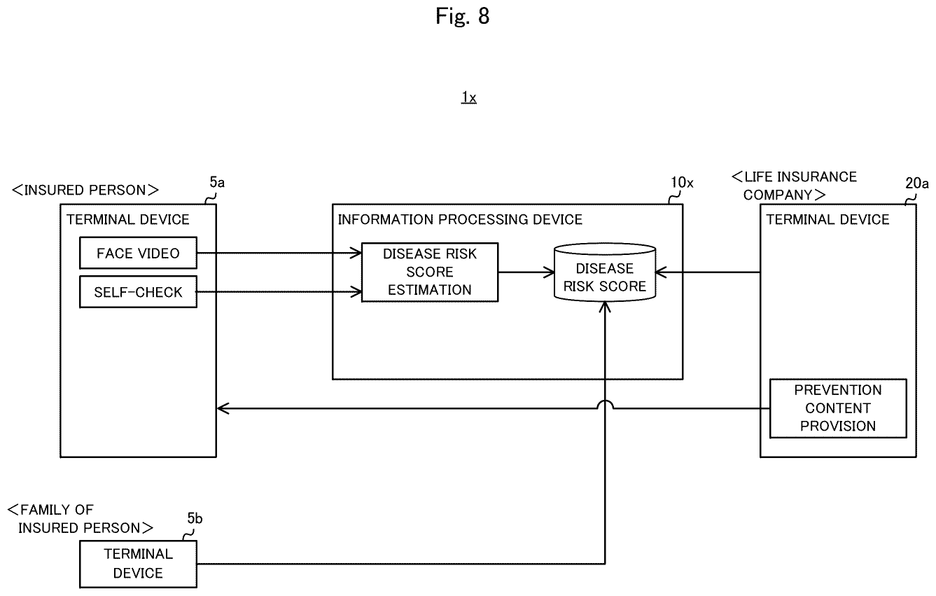

FIG. 8 is a diagram conceptually illustrating a health management system 1x according to a fifth modification. FIG. 8 includes a terminal device 5a of the insured person, a terminal device 5b of the family, an information processing device 10x, and a terminal device 20a of the life insurance company. It is assumed that a program of a self-care application provided by the information processing device 10x is installed in the terminal device 5a of the insured person and the terminal device 5b of the family.

The insured person captures a face video through the self-care application and transmits the face video to the information processing device 10x. The insured person may perform self-check via the self-care application and transmit the answer to the information processing device 10x. The self-check includes, for example, a self-check related to a cognitive function.

The information processing device 10x calculates the disease risk score based on the face video of the insured person. Then, the information processing device 10 stores the disease risk score in the DB in association with the insured person ID. In a case where there is an answer to the self-check, for example, the information processing device 10 may add a weight set according to the content of the self-check to the disease risk score determined from the face video of the insured person, and calculate the final disease risk score of the insured person.

The person in charge of the life insurance company accesses the information processing device 10 via the terminal device 20a and refers to the disease risk score of the insured person. The person in charge of the life insurance company decides content suitable for the insured person based on the disease risk score. The person in charge of the life insurance company provides the decided content to the terminal device 5a.

The disease risk score of the insured person and the family member thereof can also be referred to via the terminal devices 5a and 5b. As a result, the family member of the insured person can grasp the health condition of the insured person.

### Second Example Embodiment

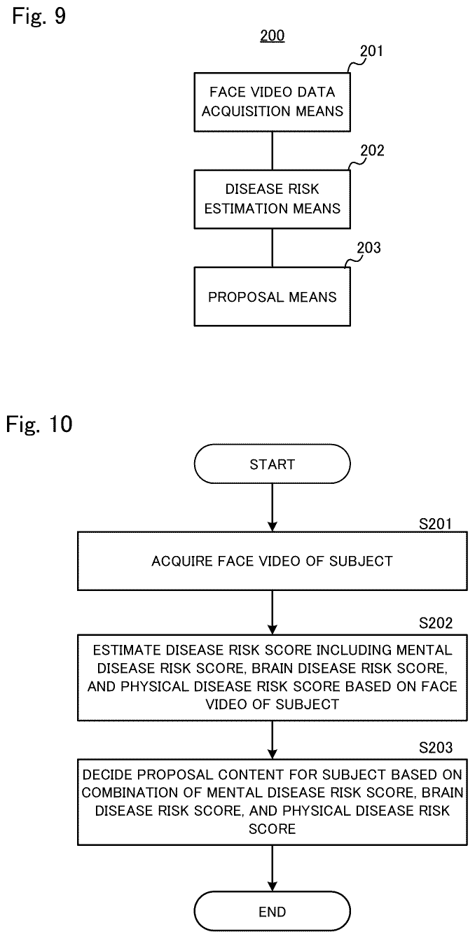

FIG. 9 is a block diagram illustrating a functional configuration of an information processing device according to a second example embodiment. An information processing device 200 includes a face video data acquisition means 201, a disease risk estimation means 202, and a proposal means 203.

FIG. 10 is a flowchart of processing by the information processing device according to the second example embodiment. The face video data acquisition means 201 acquires a face video of the subject (step S201). The disease risk estimation means 202 estimates a disease risk score including a mental disease risk score, a brain disease risk score, and a physical disease risk score based on the face video of the subject (step S202). The proposal means 203 decides a proposal content for the subject based on a combination of the mental disease risk score, the brain disease risk score, and the physical disease risk score (step S203).

According to the information processing device 200 of the second example embodiment, it is possible to make an optimal proposal according to the health condition of each person. As a result, the information processing device 200 can provide optimized services and products to the subject.

A part or all of the example embodiments described above may also be described as the following supplementary notes, but not limited thereto.

### Supplementary Note 1

An information processing device comprising:
a face video data acquisition means for acquiring a face video of a subject;
a disease risk estimation means for estimating a disease risk score including a mental disease risk score, a brain disease risk score, and a physical disease risk score based on a face video of the subject; and
a proposal means for deciding a proposal content for the subject based on a combination of the mental disease risk score, the brain disease risk score, and the physical disease risk score.

### Supplementary Note 2

The information processing device according to supplementary note 1, further comprising an insurance premium calculation means for calculating an insurance premium of the subject based on the disease risk score.

### Supplementary note 3

The information processing device according to supplementary note 1, comprising a storage means for storing past data including a past face video of the subject and a past disease risk score, wherein
the disease risk estimation means predicts a future disease risk score of the subject by using a first machine learning model configured to receive past data as an input and output a future disease risk score, and
the proposal means decides a proposal content for the subject based on the future disease risk score.

### Supplementary Note 4

The information processing device according to supplementary note 1, wherein
the disease risk estimation means estimates a value of a health item of the subject based on a face video of the subject, and decides the disease risk score based on the estimated value of the health item, and
the health item includes drowsiness, a concentration level, stress, a cognitive function, and a vital sign.

### Supplementary Note 5

The information processing device according to supplementary note 4, wherein
the mental disease risk score is decided based on at least one item of drowsiness, a concentration level, and stress of the subject,
the brain disease risk score is decided based on at least one item of drowsiness, a concentration level, and a cognitive function of the subject, and
the physical disease risk score is decided based on at least one item of drowsiness or a vital sign of the subject.

### Supplementary Note 6

The information processing device according to supplementary note 4, wherein the disease risk estimation means estimates a disease risk score of the subject by using a second machine learning model configured to receive a value of a health item as an input and output a disease risk score.

### Supplementary Note 7

The information processing device according to supplementary note 2, wherein the insurance premium calculation means reviews an insurance premium based on a past disease risk score and a current disease risk score.

### Supplementary Note 8

The information processing device according to supplementary note 1, wherein
the proposal content is a product or a service leading to health maintenance or health promotion of the subject, and
the proposal means transmits the proposal content to a terminal device of the subject.

### Supplementary Note 9

An information processing method executed by a computer, the method comprising:
acquiring a face video of a subject;
estimating a disease risk score including a mental disease risk score, a brain disease risk score, and a physical disease risk score based on a face video of the subject; and
deciding a proposal content for the subject based on a combination of the mental disease risk score, the brain disease risk score, and the physical disease risk score.

### Supplementary Note 10

A program causing a computer to execute:
acquiring a face video of a subject;
estimating a disease risk score including a mental disease risk score, a brain disease risk score, and a physical disease risk score based on a face video of the subject; and
deciding a proposal content for the subject based on a combination of the mental disease risk score, the brain disease risk score, and the physical disease risk score.

While the present disclosure has been described with reference to example embodiments and examples thereof, the present disclosure is not limited to the above example embodiments and examples. Various modifications that can be understood by those of ordinary skill in the art can be made to the configuration and details of the present disclosure within the scope of the present disclosure.

### DESCRIPTION OF SYMBOLS

1 Health Management System
5,20 Terminal Device
10 Information Processing Device
15 Database (DB)
101 Face Video Data Acquisition Unit
102 Disease Risk Estimation Unit
103 Proposal Unit
104 Insurance Premium Calculation unit

1. An information processing device comprising:
at least one memory configured to store instructions; and
at least one processor configured to execute the instructions to:
acquire a face video of a subject;
estimate a current disease risk score based on the face video of the subject;
store past data including a past face video of the subject and a past disease risk score;
predict a future disease risk score of the subject by using a first machine learning model configured to receive past data as an input and output a future disease risk score; and
decide a proposal content for the subject based on either the current disease risk score or the future disease risk score,
wherein the disease risk score includes a mental disease risk score, a brain disease risk score, and a physical disease risk score,
and wherein the proposal content is decided based on a combination of the mental disease risk score, the brain disease risk score, and the physical disease risk score included in either the current disease risk score or the future disease risk score.

2. The information processing device according to claim 1, wherein the one or more processors are further configured to calculate an insurance premium of the subject based on the current disease risk score.

3. The information processing device according to claim 1, wherein
the one or more processors estimate a value of a health item of the subject based on a face video of the subject, and decides the disease risk score based on the estimated value of the health item, and
the health item includes drowsiness, a concentration level, stress, a cognitive function, and a vital sign.

4. The information processing device according to claim 3, wherein
the mental disease risk score is decided based on at least one item of drowsiness, a concentration level, and stress of the subject,
the brain disease risk score is decided based on at least one item of drowsiness, a concentration level, and a cognitive function of the subject, and
the physical disease risk score is decided based on at least one item of drowsiness or a vital sign of the subject.

5. The information processing device according to claim 3, wherein the one or more processors estimate a disease risk score of the subject by using a second machine learning model configured to receive a value of a health item as an input and output a disease risk score.

6. The information processing device according to claim 2, wherein the one or more processors review an insurance premium based on a past disease risk score and a current disease risk score.

7. The information processing device according to claim 1, wherein
the proposal content is a product or a service leading to health maintenance or health promotion of the subject, and
the one or more processors transmit the proposal content to a terminal device of the subject.

8. An information processing method executed by a computer, the method comprising:
acquiring a face video of a subject;
estimating a current disease risk score based on the face video of the subject;
storing past data including a past face video of the subject and a past disease risk score;
predicting a future disease risk score of the subject by using a first machine learning model configured to receive past data as an input and output a future disease risk score; and
deciding a proposal content for the subject based on either the current disease risk score or the future disease risk score,
wherein the disease risk score includes a mental disease risk score, a brain disease risk score, and a physical disease risk score,
and wherein the proposal content is decided based on a combination of the mental disease risk score, the brain disease risk score, and the physical disease risk score included in either the current disease risk score or the future disease risk score.

9. A non-transitory computer readable recording medium storing a program, the program causing a computer to execute processing of:
acquiring a face video of a subject;
estimating a current disease risk score based on the face video of the subject;
storing past data including a past face video of the subject and a past disease risk score;
predicting a future disease risk score of the subject by using a first machine learning model configured to receive past data as an input and output a future disease risk score; and
deciding a proposal content for the subject based on either the current disease risk score or the future disease risk score,
wherein the disease risk score includes a mental disease risk score, a brain disease risk score, and a physical disease risk score,
and wherein the proposal content is decided based on a combination of the mental disease risk score, the brain disease risk score, and the physical disease risk score included in either the current disease risk score or the future disease risk score.
